(19) United States
(12) Patent Application Publication
(10) Pub. No.: US 2011/0313245 A1
(43) Pub. Date: Dec. 22, 2011

(54) MODULAR ENDOSCOPE

(75) Inventors: Werner Scholly, Denzlingen (DE); Stefan Schlenker, Freiburg (DE)

(73) Assignee: SCHOLLY FIBEROPTIC GMBH, Denzlingen (DE)

(21) Appl. No.: 13/148,775

(22) PCT Filed: Feb. 11, 2010
(86) PCT No.: PCT/EP10/00907

§ 371 Date: Aug. 10, 2011

(30) Foreign Application Priority Data
Feb. 11, 2009 (DE) 10 2009 008 427.4

(51) Int. Cl. A61B 1/04; A61B 1/018

(52) U.S. Cl. 600/104; 600/109

(57) ABSTRACT

An endoscope (29) having a handle (1) in which a light source (2) and a video camera (3) are directly integrated, and additional endoscope components can be coupled via a quick-coupling (5). The additional endoscope components can be probes, catheters or control elements, for example, that can be modularly combined into an endoscope.

### BACKGROUND

The invention relates to an endoscope for medical or industrial application.

Such endoscopes are known in many different constructions. For example, an endoscope with a control device is known in which a light source is arranged. The light source is connected by an optical fiber to the handle of the endoscope on which a probe can be coupled. In addition, in the control device, a video camera could be arranged that receives the image data of the probe via a second optical fiber connection leading to the handle. Especially for medical examinations, a catheter that comprises additional functions, such as a channel for rinsing fluid or compressed air, could be provided in the probe.

Accordingly, the known endoscopes have many parts and are complicated in construction and production. Because the known endoscopes are usually designed for a narrowly defined application sector, several endoscopes usually must be kept on hand for different use cases. Due to their complex construction, the known endoscopes must be cleaned and serviced when necessary only with correspondingly high expense. In addition, for conventional endoscope systems it is disadvantageous that working with these systems must be performed using the arm due to reasons of weight, which also makes precise work difficult.

### SUMMARY

Therefore, the objective is to create an endoscopic system that is significantly easier in handling and in use in comparison to the known prior art and can be used for multiple purposes.

The solution of meeting this objective is provided by the invention.

The endoscopic system according to the invention is constructed as a modular assembly set that comprises at least three of the system components that can be replaced and/or combined with each other and will be described in more detail. The individual system components can be assembled according to the use case and combined with each other. Because the system components that can be combined with each other to form an endoscope are connected detachably, if necessary, individual system components could also be replaced, for example, also for maintenance or repair purposes.

In the endoscope according to the invention, an electrical light source, advantageously a light-emitting diode and a video camera are arranged in the handle of the endoscope. Here, according to requirements, high-power LED's could also be used, for example, which emit light of different wavelengths. Instead of expensive, less flexible, heavy, and fragile optical fibers, only electrical current and signal cables are required between the handle and a control device. Therefore, the force acting through the cable on the handle also decreases and thus also the counter force that must be applied by the user for compensation. The endoscope according to the invention simplifies the work for the user just with his or her fingers and promotes high precision of the work that is performed. It is further advantageous that optical losses that can occur due to multiple coupling of the optical fiber in conventional endoscopes are avoided in the endoscope according to the invention. Thus, significantly easier work with the endoscope according to the invention is possible. Because no space is needed for optical fiber connections, the handle can be smaller and lighter overall. Therefore, the endoscope can be held more easily by hand and guided with fingertips. This allows significantly more precise control of the image section and fatigue-free work.

The probe of the endoscope can have a multipurpose construction and can have, for example, a flexible or rigid shaft whose length can vary according to application. The optics in the probe tip for capturing the image could be arranged straight or at an angle for allowing different viewing angles.

In a preferred development of the invention, the endoscope also has a control for deflection and/or turning the catheter tip or probe tip and the control is arranged separately or in combination with other functional units in an endoscope component.

Through the control, the tip of a flexible probe or of a flexible catheter could be angled, so that the viewing field can be changed. This control is useful especially for probes with straight optics, because in the case of oblique optics, changing the viewing field is also possible by turning the endoscope about the longitudinal axis. Nevertheless, the control is not limited to such probes.

The individual functional units of the endoscope according to the invention can be arranged individually or in combination on different endoscope components. In this way, a modular endoscope system is produced that can be equipped with only the necessary functions according to the application and requirements by easily assembling the corresponding endoscope components using quick-connect couplings. According to the application, individual functional units could also be arranged on different endoscope components.

In one construction of the invention, one of the endoscope components has a handle on whose quick-connect coupling a probe component could be coupled and the video camera has an optical interface for the probe that can be coupled on the quick-connect coupling.

In one alternative construction, the endoscope component has a handle in which a probe and a control for deflection the probe tip are integrated, wherein the camera is arranged on the free probe tip. Because the video camera is arranged on the probe tip, the image is captured directly at the location of observation. Therefore, images do not need to be transmitted through image guides within the probe, which significantly reduces the optical requirements on the probe. In this way, the endoscope is overall more robust and less susceptible to noise. In addition, the optical interfaces on the quick-connect coupling are eliminated, which also makes the endoscope more cost-effective.

Alternatively, one of the endoscope components could have a handle with two independent electrical light sources in which a probe is integrated and the camera is arranged on the free probe tip and the probe part projecting from the handle can be adjustable in length. The second light source could have, for example, a different wavelength, for example, in the ultraviolet or infrared range. In this way, for example, fluorescing dyes in tissue could be excited or composite materials could be cured. The second light source could also be a laser with which, for example, tissue could be coagulated.

For the combination with a probe-less handle, one of the endoscope components has a probe. This probe component can be connected by the quick-connect coupling to a handle component and has optical interfaces for the camera and the at least one light source. This probe could also be produced favorably as a single-use product, because this probe does not contain its own optics, but instead only, for example, optical fibers for forwarding the LED's or similar light sources located in the handle.

According to the application of the endoscope, it could be advantageous if the probe component has a control for deflection the probe tip. Especially for straight probe optics and/or very narrow insertion channels, the viewing field can be easily changed without the endoscope having to be greatly moved as a whole.

It can furthermore be advantageous if the control for a catheter or a probe is arranged in a separate control component.

In one construction of the invention, one of the endoscope components has a catheter with a tip that can be controlled by a control that can be coupled.

Alternatively, one of the endoscope components could have a catheter with an integrated control or one of the endoscope components has a catheter that can be controlled indirectly by an inserted probe.

In addition, it could be preferable if the catheter has additional functional units, such as grippers or similar manipulators or additional lumens, for example, for passing fluids or for connecting to different light sources.

In the endoscope according to the invention, several endoscope components can be combined in a modular way to form an endoscope. Several endoscope components could also be contained in an assembly set for an endoscope from which, according to the application, a matching endoscope could be assembled.

In medical applications, the probes and/or catheters are in direct contact with the body and therefore must be absolutely sterile. Therefore it can be advantageous if the endoscope components are constructed with probes and/or catheters for single use. In this way, cleaning and sterilization after use can be eliminated. For patients, the risk of infection is therefore reduced. Also through the modular construction of the endoscope according to the invention, the single-use component could be created so that it contains no expensive and complicated functional units and thus can be produced cost-effectively.

### BRIEF DESCRIPTION OF THE DRAWINGS

The invention is explained in detail below using a few preferred embodiments with reference to the accompanying drawings.

Shown are:

FIG. 1 a construction of an endoscope handle,

FIG. 2 a second construction of an endoscope handle with integrated probe and control,

FIG. 3 another construction of an endoscope handle with probe that is adjustable in length,

FIG. 4 a probe part that belongs to an endoscopic assembly set with a modular construction,

FIG. 5 another probe part with integrated control,

FIG. 6 an endoscope component with a control for a catheter that can be coupled,

FIG. 7 a catheter with integrated control,

FIG. 8 a catheter that can be controlled by an external control component,

FIG. 9 a catheter that can be controlled only by an inserted probe,

FIG. 10 a catheter with two optical interfaces for different light sources,

FIGS. 11 and 12 other catheters that can be controlled by an inserted probe,

FIGS. 13-17 endoscopes each of which is combined from different endoscope components.

### DETAILED DESCRIPTION OF THE PREFERRED EMBODIMENTS

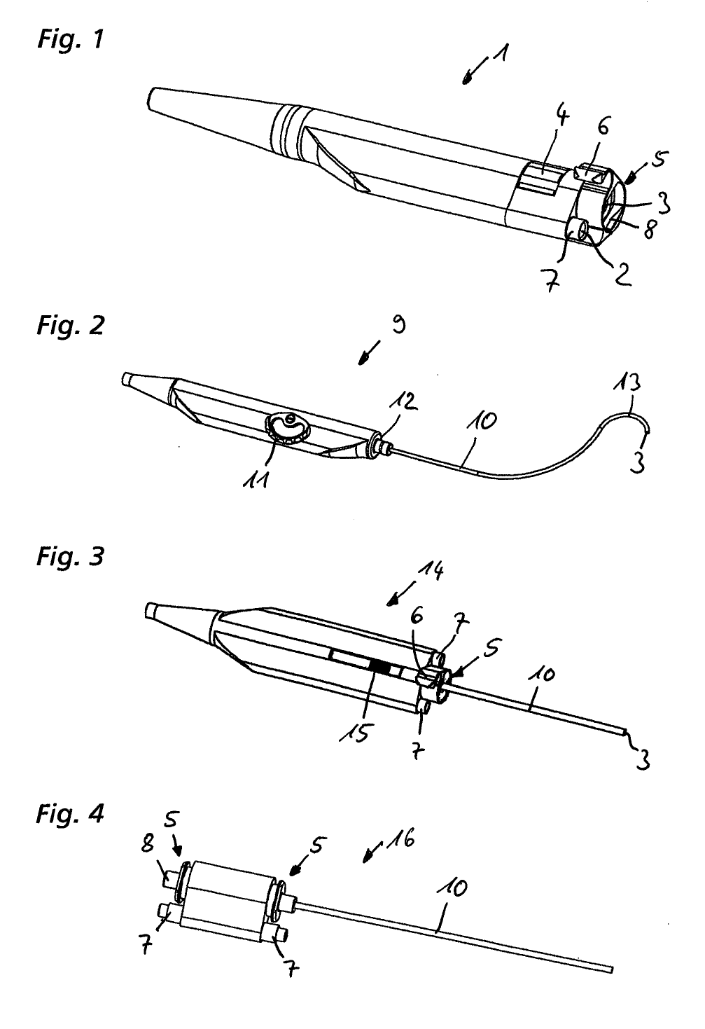

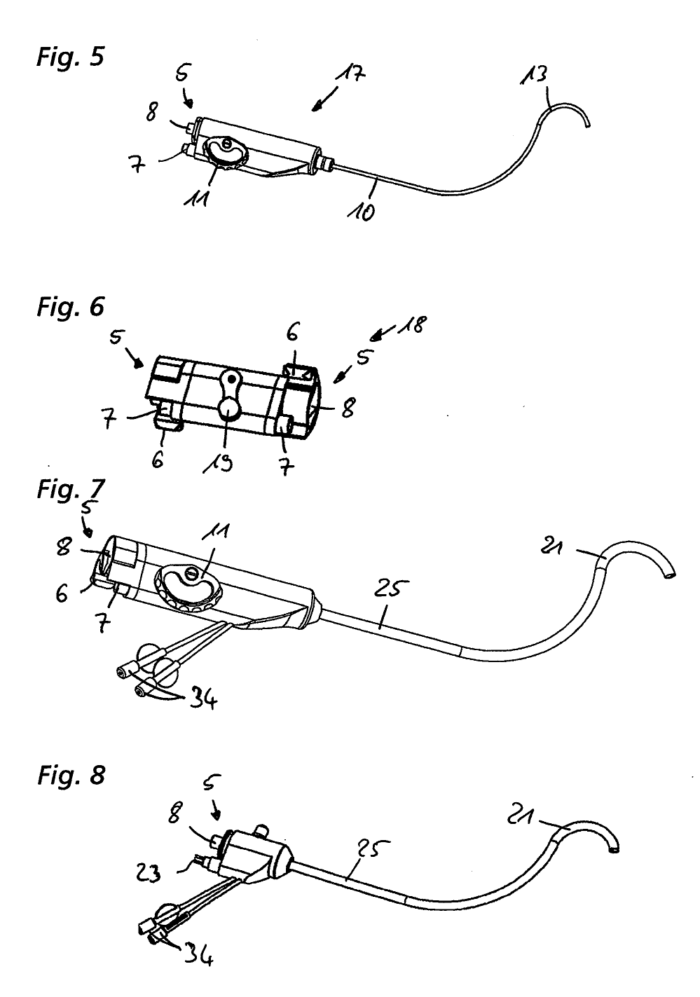

In FIGS. 1 to 12, components of an endoscopic system are shown that is constructed as a modular assembly set. The assembly set presented here comprises, for example, the three handles 1, 9, and 14 shown in FIGS. 1 to 3. In addition, the assembly set also provides the probe parts 16 and 17 shown in FIGS. 4 and 5.

The handle 1 shown in FIG. 1 has an electrical light source 2, for example, an LED. Furthermore, a video camera 3 in the form of a camera chip, for example, a CCD or CMOS chip, is integrated in the handle 1. The camera chip 3 is supported displaceable within the handle for focusing through the use of a hand wheel 4 in the longitudinal direction.

For coupling additional endoscope components, the handle 1 has available a quick-connect coupling 5 with a foldable lock 6. For transferring the light signals, an optical interface 7 is arranged in the area of the light source 2. Likewise, in the area of the camera 3, another optical interface 8 is provided for transferring image information.

Therefore, because the light source 2 and the camera 3 are integrated in the handle 1, for operation only electrical current and signal cables are needed, wherein the handling of the endoscope is significantly improved and simplified.

FIG. 2 shows an alternative construction of a handle according to the invention designated with 9. In the handle 9, like in the handle 1 of FIG. 1, an electrical light source 2 is integrated. In addition, the handle has a flexible probe 10 and a control for the probe that can be operated by a hand wheel 11. Through use of the hand wheel 11, the probe tip 13 can be angled. At the probe end of the handle 9, a coupling 12 is arranged for coupling a catheter.

In this construction, the camera 3 is arranged at the probe tip 13. This has the advantage that only the light from the light source 2 must be guided through the probe 10 to the probe tip 13. The requirements on the optical fiber are therefore significantly lower than for image transmission. The probe 10 therefore can be produced significantly more cost-effectively and is more robust.

In FIG. 3, another handle 14 is shown in which a flexible probe 10 is already integrated similar to the handle 9 of FIG. 2. Here, the camera 3 is also arranged on the probe tip 13. In addition, the probe 10 is adjustable in length by an adjustment mechanism that can be operated by a slide 15. In contrast, a control for the deflection adjustment is missing.

In comparison to the handle 1 of FIG. 1, this handle 14 has two independent, electrical light sources 2 that have available their own optical interface 7 on the coupling 5.

The second light source 7 can contain, for example, a different wavelength range, in order to create a contrast image or to excite special fluorescing dyes (UV light). A laser for manipulating tissue could also be provided.

In FIG. 4, a probe component 16 is shown that can be combined, in principle, with the handle 1 of FIG. 1. The probe component 16 has, on two sides, quick-connect couplings 5 for coupling on other endoscope components, wherein optical interfaces 7 for the light source 2 are provided on both couplings. The probe 10 likewise has optical interfaces 8 for transmitting image information to the camera 3 of the handle 1.

FIG. 5 shows another probe component 17 with a flexible probe 10 and an integrated control for the probe tip 13. Through use of a side hand wheel 11, the probe tip 13 can be angled. A quick-connect coupling 5 with optical interfaces 7, 8 allows the coupling of a handle component.

FIG. 6 shows a control component 18 that has, on both sides, quick-connect couplings 5 for coupling additional endoscope components. The component has only one control with a hand lever 19 for controlling a catheter that can be coupled.

FIG. 7 shows a catheter 20 that can be coupled as an endoscope component on a matching handle or another endoscope component. The catheter 20 has a quick-connect coupling 5 that has two optical interfaces 7, 8. Furthermore, the catheter 20 provides a control with which the catheter tip 21 can be angled via a hand wheel 11. The catheter 20 further has a shaft 25 in which a probe 10 can be introduced.

In addition, the catheter provides two working channels 34 by which, for example, compressed air, fluid, vacuum, or similar medium could be guided to the catheter tip 21.

The catheter 22 that is shown in FIG. 8 has no integrated control in comparison to the catheter 20 of FIG. 7. Instead, the catheter 22 has a control interface 23 that can be connected, for example, to an external control 18 according to FIG. 6. In this way, the catheter tip 21 can also be angled for this catheter 22. If the application requires no control, then the catheter 22, however, also can be operated without a control function.

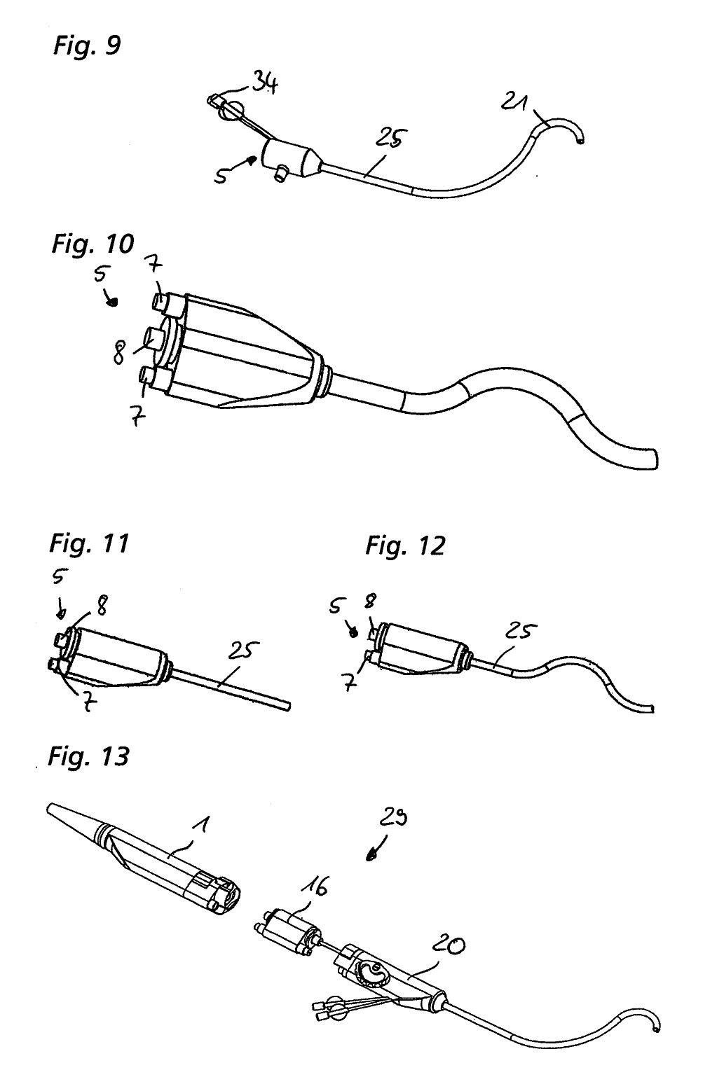

The catheter 24 of FIG. 9 has no direct control function. A control of the catheter tip 21 is possible in this endoscope component 24 via a controllable probe 10 that can be inserted into the catheter shaft 25.

The catheter component 26 of FIG. 10 has optical interfaces 7 for two separate light sources 2 and can be coupled, in particular, with the handle 14 of FIG. 3.

FIGS. 11 and 12 show additional catheter components 27 and 28 that can be used as an alternative to the catheter 26, but that have only one optical interface 7 for a light source 2.

The endoscope components shown in FIGS. 1 to 12 form, in practice, one endoscope assembly set from which various endoscopes can be assembled in a modular manner. Here, components fitting the requirements of the application can be selected so that an optimum endoscope system is available at any time. Here, only as examples, the components shown here stand for a plurality of other possible components.

Furthermore it is possible to equip existing endoscopes with new or additional functionality, for example, many existing, older probes and/or catheters could be used with a handle according to the invention and thus profit from the integrated camera and lighting technology.

In FIGS. 13 to 17, as examples, different endoscopes are shown that can be combined from the endoscope components shown above.

FIG. 13 shows an endoscope that is designated overall with 29 and is combined from a handle 1, a probe part 16, and a catheter 20. The connection of the individual components 1, 16, and 20 is here realized by make-contact/coupling spring systems. The deflection of the catheter tip of the catheter 20 is carried out by the integrated operating element integrated in the catheter, wherein the flexible probe of the probe part 16 inserted up to the tip in the catheter 20 is also carried along. While a camera and a light source are integrated in the handle 1, the probe part 16 has flexible optics. Through the operating element integrated in the catheter 20, the catheter tip can be moved with the probe containing flexible optics into the desired viewing field. The catheter 20 is constructed as a flexible, multi-lumen tube that can also have suction/rinsing tube connections.

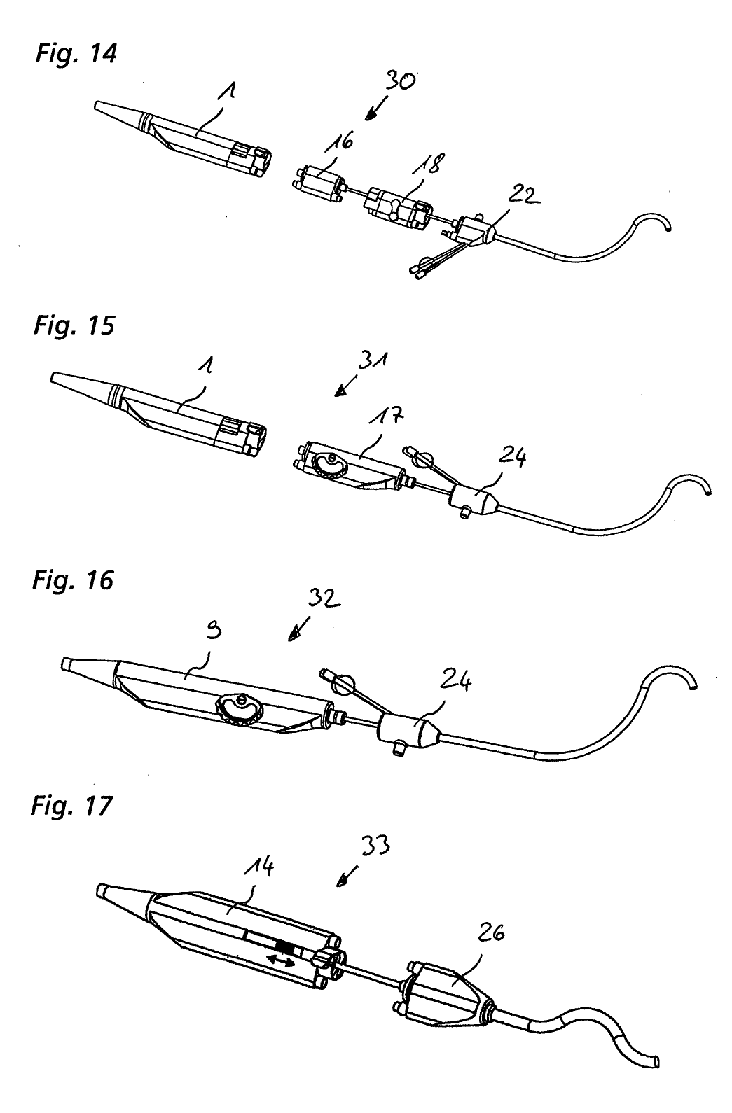

The endoscope 30 of FIG. 14 includes a handle 1, a probe part 16, a control adapter 18, and a catheter 22. Also for the endoscope 30 shown in FIG. 14, the connection of the individual components 1, 16, 18, and 22 is performed by a make contact/coupling spring system. The angling of the catheter tip of the catheter 22 is performed by the operating element located on the control adapter 18, wherein the flexible probe inserted through the control adapter 18 up to the tip in the catheter 22 is also moved along. While a camera and also a light source are also integrated in the handle 1, the probe part 16 has flexible optics. The control adapter provided for controlling the catheter 22 is connected detachably to the catheter 22 at one of its ends and to the probe part 16 at its other catheter end. The probe projecting on the probe part 16 is guided through a corresponding guide opening in the control adapter 18 and inserted into the sleeve-shaped catheter of the catheter part 22. The catheter part 22 of the endoscope 30 shown here also has a flexible, multi-lumen tube that could also have suction/rinsing tube connections here.

In FIG. 15, an endoscope 31 is shown that is assembled from a handle 1, a probe part 17, and a catheter part 24. In this variant, the endoscope 31 includes the handle 1, the probe part 17 having an integrate control, and the catheter part 24. The connection of the individual components 1, 17, and 24 is also realized here by a make-contact/coupling spring system. The angling of the catheter tip provided on the catheter part is performed by the probe of the probe part 17 inserted into the sleeve-shaped catheter of the catheter part 24, wherein the probe can be angled by the operating element integrated on the probe part 17 and thus the catheter part also moves along. In the handle 1, the camera and light source are also integrated. The catheter part 24 has a multi-lumen tube as a catheter that can be connected to laterally projecting suction/rinsing tube connections.

In FIG. 16, a handle 9 is combined with a catheter part 24 to form an endoscope 32. In this variant that forms the endoscope 32 includes the handle 32 with the integrated, controllable probe and the catheter part 24. The deflection of the catheter tip is performed by the probe that is inserted into the catheter and can be deflected by the integrated operating element and thus the catheter tip also deflects. Here, the camera chip provided in the handle 9 is arranged in the free end area of the probe projecting on the handle 9.

The endoscope 33 of FIG. 17 combines the handle 14 with a catheter part 26 and is thus suitable for the application of two different light sources 2. For the endoscope 33 which includes the handle 14 with probe and the catheter part 26 set on the probe of the handle 14, the connection of the components is likewise performed by a make-contact/coupling spring system. Through the two fiber-optic interfaces provided on the handle 14, for one, the light needed for illumination of the viewing field can be coupled in and also laser light or fluorescent light, for example, could be coupled in, if necessary. The probe projecting on the handle 14 can here be regulated in length by a displaceably guided pushbutton that is provided on the side of the handle 14. Here, the endoscope 33 shown in FIG. 17 allows light and image to be clearly separated from each other.

The endoscope 33 could be used alternatively also in connection with the catheter parts 27 or 28, but then only with one light source 2.

1. A modular endoscopic system that comprises at least three of the following system components that can be exchanged and combined with each other, including at least one handle (1, 9, 14) as a first system component that has a coupling part with at least one fiber-optic interface for light transmission and at least one camera chip for image capture, the handle being selected from:
a first handle (1) having the coupling part that is connectable to a coupling counter part of a probe part having a probe that has flexible optics for image transmission,
a second handle (14) with an integrated probe that is held moveable in the second handle such that a length of a probe sub-area projecting past the second handle (14) is variable, wherein the integrated probe carries the camera chip on a free probe end area, and
a third handle (9) with an integrated probe that can be pivoted or controlled at least on a free probe end area projecting past the coupling part of the handle (9) by a control integrated in the handle (9), wherein the integrated probe carries the camera chip (32) on the free probe end area,
the other system components being selected from:
a probe part (16, 17) that has a first coupling counter part on a first side thereof that is detachably coupleable on the coupling part of the handle and a second coupling counter part on a second side thereof carrying a probe that is detachably coupleable on a coupling part of a catheter or a control adapter,
a catheter part (20) that is detachably coupleable with a coupling part thereof on a coupling counter part of a probe and a catheter thereof holding the probe of the probe part that is controllable or pivotable at least in a free end area by a control integrated in the catheter part,
a catheter part (22) that is detachably coupleable with a coupling part thereof on a coupling counter part of a control adapter and the catheter part carries a catheter that is controllable or pivotable at least in a free catheter end area by a control provided on the control adapter,
a control adapter (18) that has a first coupling part on a first side thereof that is detachably coupleable with a coupling counter part of a probe part and a second coupling part on a second side thereof that is detachably coupleable with a coupling counter part of a catheter part, wherein the control adapter has a control for controlling at least a free catheter end area of the catheter part coupled on the control adapter,
a catheter part (24) that is detachably coupleable with a coupling part thereof to a handle, the catheter holds, in itself, a probe that is integrated in the handle and projects with a probe end area past the coupling part,
a catheter part (24) having a catheter holding a probe of a probe part, the catheter has a flexible construction at least in a free end area such that the catheter is movable by the probe held by the catheter and controllable by a control provided on the probe part, and
a probe part (26) that has a coupling counter part that is detachably coupleable on the coupling part of a handle and in whose probe a camera probe can be inserted that carries a camera chip on a free end and held on the handle.

2. An endoscope having a modular construction comprising at least two endoscope components that are coupleable to each other by quick-connect couplings (5) and has at least the following functional units: a handle with at least one electrical light source (2) and a video camera (3), wherein the light source (2) has an optical interface (7) on one of the quick-connect couplings (5), a probe (10) and a catheter (25) that is adapted to be pushed onto the probe (10), wherein the functional units are arranged individually or in combination in the endoscope components.

3. The endoscope according to claim 2, wherein the endoscope has a control for deflecting and/or turning a catheter tip (21) or probe tip (13) and the control is arranged separately or in combination with other functional units in one of the endoscope component.

4. The endoscope according to claim 2, wherein one of the endoscope components includes the handle (1) on whose quick-connect coupling (5) a probe component (16) can be coupled and the video camera (3) has an optical interface (8) for the probe (16) that can be coupled on the quick-connect coupling (5).

5. The endoscope according to claim 2, wherein one of the endoscope components includes the handle (9) in which a probe (10) and a control element (11) are integrated for deflecting a free probe tip (13) and the camera (5) is arranged on the free probe tip (13).

6. The endoscope according to claim 2, wherein one of the endoscope components includes the handle (14) which has two independent electrical light sources (2) in which a probe (10) is integrated, the camera (3) is arranged on a free probe tip (13), and the probe (10) projecting from the handle (14) is adjustable in length.

7. The endoscope according to claim 2, wherein one of the endoscope components has the probe (10).

8. The endoscope according to claim 2, wherein the probe component (17) has a control (11) for deflecting a probe tip (13).

9. The endoscope according to claim 2, wherein one of the endoscope components (18) has only one control (19) for a catheter that is adapted to be coupled thereto.

10. The endoscope according to claim 2, wherein one of the endoscope components has a catheter (22) with a tip (21) that can be controlled by a control (18) that is adapted to be coupled thereto.

11. The endoscope according to claim 2, wherein one of the endoscope components has a catheter (20) with an integrated control.

12. The endoscope according to one of claims 1 or 11, characterized in that one of the endoscope components has a catheter (24) that is controllable indirectly by an inserted probe.

13. The endoscope according to claim 2, wherein the catheter has additional functional units (34), comprising grippers or similar manipulators.

14. The endoscope according to claim 2, wherein several of the endoscope components can be combined in a modular way to form the endoscope.

15. The endoscope according to claim 2, wherein the endoscope (29) has a modular construction made from the handle (1), a probe (16) integrated into the handle for angling a free probe tip (13), with the camera (5) arranged on the free probe tip (13), and a catheter (20) with an integrated control.

16. The endoscope according to claim 2, wherein the endoscope (30) has a modular construction made from the handle (1), a probe (16) with the video camera (3) having an optical interface (8) for the probe (16) that can be coupled on the quick-connect coupling (5), a control (18) for angling a probe tip (13), and a catheter (22).

17. The endoscope according to claim 2, wherein the endoscope (31) has a modular construction made from the handle (1), a probe (17), and a catheter (24) with an integrated control.

18. The endoscope according to claim 2, wherein the endoscope (32) has a modular construction made from the handle (9) on whose quick-connect coupling (5) a probe component (16) can be coupled and the video camera (3) has an optical interface (8) for the probe (16) that can be coupled on the quick-connect coupling (5) and a catheter (24) with an integrated control.

19. The endoscope according to claim 2, wherein the endoscope (33) has a modular construction made from the handle (14) in which a probe (10) and a control (11) are integrated for angling a free probe tip (13) and the camera (5) is arranged on the free probe tip (13) and a catheter (26) with an integrated control.
